(19) United States
(12) Patent Application Publication
(10) Pub. No.: US 2022/0137028 A1
(43) Pub. Date: May 5, 2022

(54) In Vitro Assays as Indicator of Local Tolerance

(71) Applicant: Lonza Ltd, Visp (CH)

(72) Inventors: Peter Bruegger, Anwil (CH); Dieter Roethlisberger, Muttenz (CH); Hanns-Christian Mahler, Loerrach (DE)

(21) Appl. No.: 17/433,252

(22) PCT Filed: Feb. 2, 2020
(86) PCT No.: PCT/EP2020/054537

§ 371 Date: Aug. 24, 2021

Related U.S. Application Data
(60) Provisional application No. 62/810,242, filed on Feb. 25, 2019.
(60) Provisional application No. 62/831,921, filed on Apr. 10, 2019.

(30) Foreign Application Priority Data
Mar. 22, 2019 (EP) 19 164 698.3
May 14, 2019 (EP) 19 174 443.2
Sep. 30, 2019 (EP) 19200512.2

(51) Int. Cl. G01N 33/50; G01N 33/86

(52) U.S. Cl. CPC G01N 33/5088; G01N 33/86

(57) ABSTRACT

The present disclosure is directed to in vitro methods for determining local tolerance. In some embodiments, the in vitro method is a miscibility test.

### FIELD OF INVENTION

The present invention is directed to in vitro methods for determining local tolerance of the administration of a liquid drug product to an animal. In some embodiments, the in vitro method is a hemolysis test. In some embodiments, the in vitro method is a miscibility test.

### BACKGROUND

Safe and effective delivery of active pharmaceutical ingredients (APIs) is an important consideration when formulating drug products. Injectable subcutaneous, intravenous, and/or intramuscular pharmaceutical formulations may include non-aqueous solvents due to poor water solubility of the API. Non-aqueous solvents, while capable of solubilizing the API, may have undesirable pharmacological and even toxicological effects in the patient, e.g., at the site of injection. Local tolerance testing is an important component in assessing drug product safety. Medicines that are not well tolerated locally not only can cause a variety of conditions at or near the site of administration, including thrombophlebitis, but also can potentially cause systemic bodily harm, such as particulate buildup in the bloodstream, which, in a severe case, could lead to an embolism.

### SUMMARY OF INVENTION

Some aspects of the invention are:

1. Use of an in vitro determination of blood tolerance for a substance or combination of substances to be used in a pharmaceutical composition intended for parenteral administration to an animal as a surrogate test for animal and/or human testing.
2. The use according to item 1, wherein the substance to be tested is selected from active pharmaceutical ingredients, organic solvents, co-solvents, surfactants, and combinations of two or more thereof, wherein the active pharmaceutical ingredient is preferably poorly water soluble.
3. The use according to any preceding item, wherein the animal to which the pharmaceutical composition is to be administered parenterally is a mammal, preferably selected from humans, domestic animals and productive livestock.
4. The use according to any preceding item, wherein the in vitro determination of the blood tolerance is done by a hemolysis test.
5. The use according to item 4, wherein for the hemolysis test whole blood, or heparinized blood is used.
6. The use according to any one of items 4 to 5, wherein the hemolysis test comprises determining the hemolysis in % caused by a certain substance for a plurality of concentrations of the substance, plotting a curve from the test results with hemolysis in % on the ordinate and the concentration of the substance on the abscissa, and obtaining the concentration corresponding to a predetermined hemolysis threshold value from said curve.
7. The use according to item 6, wherein the measuring of the hemolysis and/or the plotting of the curve and/or the obtaining of the concentration corresponding to the predetermined threshold hemolysis value is carried out by using an automatized system.
8. The use according to item 6 or 7, wherein the predetermined hemolysis threshold value is 10% or 5%.
9. The use according to any one of items 4 to 8, wherein the maximum possible hemolysis is determined by measuring the amount of hemoglobin by using Drabkin's solution.
10. The use according to any one of items 4 to 9, wherein the amount of hemolysis is determined by measuring absorbance at 540 nm.
11. The use according to any one of items 4 to 10, wherein the different concentrations of the substance are obtained by diluting a stock solution of the substance with or dissolving the substance in water or an aqueous NaCl solution.
12. The use according to any one of items 1 to 3, wherein the in vitro determination of the blood tolerance is done by a miscibility test.
13. The use according to item 12, wherein for the miscibility test blood plasma obtained from whole blood or heparinized blood is used.
14. The use according to any one of items 12 and 13, wherein a plurality of concentrations of the substance is tested for precipitation or coagulation in blood.
15. The use according to any one of items 12 to 14, wherein the lowest concentration when precipitation or coagulation first occurs is determined.
16. The use according to any one of items 14 or 15, wherein the plurality of concentrations is obtained by varying the mixing ratio of (a) blood plasma and (b) the substance to be tested if the substance is liquid at room temperature or a solution of the substance to be tested if the substance is a solid at room temperature.
17. The use according to any one of items 14 to 16, wherein precipitation or coagulation is detected by light scattering, a turbidity sensor, a turbidimeter, a nephelometer or a spectrophotometer.

Further aspects described herein are:

A. An in vitro method for at least partially replacing animal and/or human testing required for finding pharmaceutically acceptable concentrations of substances to be used in pharmaceutical compositions intended for parenteral administration, wherein the method comprises in vitro determination of blood tolerance for a substance or combination of substances to be used in a pharmaceutical composition intended for parenteral administration to an animal;
B. The method according to item A, wherein the substance to be tested is selected from active pharmaceutical ingredients, solvents, co-solvents, surfactants, and combinations of two or more thereof, wherein the active pharmaceutical ingredient is preferably poorly water soluble.
C. The method according to item A or B, wherein the animal to which the pharmaceutical composition is to be administered parenterally is a mammal, preferably selected from humans, domestic animals and productive livestock.
D. The method according to any of items A to C, wherein the in vitro determination of the blood tolerance is done by a hemolysis test.
E. The method according to item D, wherein for the hemolysis test whole blood, or heparinized blood is used.
F. The method according to any one of items D or E, wherein the hemolysis test comprises determining the hemolysis in % caused by a certain substance for a plurality of concentrations of the substance, plotting a curve from the test results with hemolysis in % on the ordinate and the concentration of the substance on the abscissa, and obtaining the concentration corresponding to a predetermined hemolysis threshold value from said curve.
G. The method according to item F, wherein the measuring of the hemolysis and/or the plotting of the curve and/or the obtaining of the concentration corresponding to the predetermined threshold hemolysis value is carried out by using an automatized system.
H. The method according to item F or G, wherein the predetermined hemolysis threshold value is 10% or 5%.
I. The method according to any one of items D to H, wherein the maximum hemolysis possible is determined by measuring the amount of hemoglobin by using Drabkin's solution.
J. The method according to any one of items D to I, wherein the amount of hemolysis is determined by measuring absorbance at 540 nm.
K. The method according to any one of items D to J, wherein the different concentrations of the substance are obtained by diluting a stock solution of the substance with or dissolving the substance in water or an aqueous NaCl solution.
L. The method according to any one of items A to C, wherein the in vitro determination of the blood tolerance is done by a miscibility test.
M. The method according to item L, wherein for the miscibility test blood plasma obtained from whole blood or heparinized blood is used.
N. The method according to any one of items L and M, wherein a plurality of concentrations of the substance is tested for precipitation or coagulation in blood.
O. The method according to any one of items L to N, wherein the lowest concentration when precipitation or coagulation first occurs is determined.
P. The method according to any one of items N to O, wherein precipitation or coagulation is detected by light scattering, a turbidity sensor, a turbidimeter, a nephelometer or a spectrophotometer.

In some embodiments, the present disclosure provides an in vitro method of screening a pharmaceutical formulation comprising an API, a solvent and a co-solvent or other compound of interest, or a formulated drug product that may include solvents, co-solvents, surfactants and other excipients, for hemolysis propensity, comprising: (a) contacting the formulation comprising an API, a solvent and a co-solvent or other compound of interest, or a formulated drug product that may include solvents, co-solvents, surfactants and other excipients with human or animal blood in vitro to produce a second solution; and (b) separating erythrocytes from the second solution and utilizing the inventions disclosed herein to assess local tolerance and analyze and determine appropriate concentrations of API and other compounds of interest, or to analyze and determine the composition of excipients and other components, and the appropriate concentration of the components of a formulated drug product, among other things by measuring hemolysis of the erythrocytes as a proxy for local tolerance.

In some embodiments, the present disclosure provides an in vitro method of screening a pharmaceutical formulation comprising an API and a solvent, and, in certain embodiments, also comprising a co-solvent or other excipient, or other compound of interest, or a formulated drug product that may include solvents, co-solvents, surfactants and other excipients for miscibility with plasma, comprising: (a) contacting the formulation comprising an API, a solvent and a co-solvent or other compound of interest, or a formulated drug product that may include solvents, co-solvents, surfactants and other excipients with human or animal blood, particularly blood plasma, to produce a solution; and (b) assessing precipitation in the solution, as a means to analyze and determine appropriate concentrations of a formulated drug product.

The inventions disclosed herein also may be used to determine infusion rates, among other administration regimens and strategies.

### FIGURES

FIGS. 1A-1E shows experiments described in Example 2. FIGS. 1A-1E show the percent hemolysis in a solution containing PEG 300 (FIG. 1), PEG-400 (FIG. 1B), ethanol (FIG. 1C), dimethylacetamide (DMA) (FIG. 1D), and propylene glycol (PG) (FIG. 1E) at varying concentrations diluted with either water or saline.

FIG. 2 shows percent hemolysis values in co-solvents from Example 2. WFI: water for injection; NaCl: saline; Ctrl: WFI: 0% co-solvent.

FIG. 3 Graphically shows percent hemolysis in co-solvents diluted with 0.9% NaCl, as described in Example 2. All co-solvents showed a sigmoidal curve shape. The maximum well-tolerated co-solvent concentration (corresponding to 5% hemolysis) is shown for each co-solvent.

FIG. 4 shows an embodiment of the hemolysis test described herein. Equal volumes of the test solution (e.g., drug product or “DP”) and heparinized blood (e.g., 1 mL each) are mixed in a test tube, incubated for 10 minutes at 37° C., centrifuged, and measured to determine percent hemolysis with varying concentrations of test solution (e.g., co-solvent, surfactant, or API).

FIG. 5 shows an embodiment of the miscibility test described herein. Heparinized human blood is centrifuged to produce supernatant (plasma). Various concentrations of the test solution (e.g., co-solvent, surfactant, or API) are added to the plasma. Precipitation is an indicator of lack of miscibility.

FIG. 6 shows an interpretation of the miscibility test. The tested solutions from FIG. 5 are applied to an intravenous injection. Non-precipitating solutions from the miscibility test show a reduced bolus size delivered from the injection compared with the precipitating solution.

### DETAILED DESCRIPTION OF THE INVENTION

In embodiments, the present invention provides in vitro methods for testing tolerance of a pharmaceutical formulations. The present disclosure advantageously provides simple and effective in vitro methods to test multiple solutions, e.g., with different co-solvents, surfactants, and/or active pharmaceutical ingredients (APIs), for tolerance, facilitating the formulation process. In embodiments, the methods of the invention provide simple and inexpensive methods to predict the tolerance of small molecule pharmaceutical formulations. The methods do not require animal models and efficiently screen formulations for local tolerance. By determining the maximum acceptable amounts of relevant ingredients in accordance with the present invention it is possible to avoid animal/human testings to find out such concentrations. i.e. animal/human testings with inacceptable concentrations can be avoided.

As used herein, “tolerance” refers to biocompatibility. In embodiments, tolerance and biocompatibility of a formulation is evaluated based on whether the cells and/or tissues at the site of administration (e.g., injection), or cells and/or tissues at a location other than the site of administration (e.g., if the site of administration is at one part of the body and biocompatibility or tolerance may be measured at a different part of the body), or cells and/or tissues that mimic cells and/or tissues at the site of administration, under autophagy, lysis, and/or apoptosis as a result of the formulation; whether the site of administration (e.g., injection site) is substantially free of inflammation or irritation after administration; whether the API is effectively delivered to desired cell, tissue, and/or organ; and/or whether the subject experiences discomfort or illness after administration of the formulation.

In some embodiments, the in vitro method of testing tolerance of a pharmaceutical formulation tests the hemolysis propensity of the formulation and, in other embodiments, the method can be utilized to determine the appropriate composition of such a formulation. “Hemolysis” refers to the rupture of the erythrocytes, i.e., red blood cells, and the release of their contents into surrounding fluid, e.g., plasma. Hemolysis at the site of administration, e.g., injection site, may cause local irritation and inflammation, and may also decrease the amount of API delivered to the site. In some embodiments, hemolysis occurs when red blood cells are contacted with a surfactant. In some embodiments, hemolysis occurs when red blood cells are contacted with a poorly water-soluble compound, e.g., a poorly water-soluble API.

In some embodiments, the present disclosure provides an in vitro method of screening a pharmaceutical formulation comprising an active pharmaceutical ingredient (API), a solvent and, optionally, a co-solvent, for hemolysis ability, comprising: (a) contacting the formulation with a first solution comprising human blood and a blood thinner to produce a second solution; and (b) separating erythrocytes from the second solution and monitoring hemolysis of the erythrocytes.

In some embodiments, the solvent is water. In some embodiments, the solvents comprise a salt. In some embodiments, the solvent comprises NaCl. In some embodiments, the solvent comprises between about 0.1% to about 20% NaCl. In some embodiments, the solvent comprises between about 0.5% to about 2.0% NaCl. In some embodiments, the solvent comprises about 0.1%, about 0.2%, about 0.3%, about 0.4%, about 0.5%, about 0.6%, about 0.7%, about 0.8%, about 0.9%, or about 1.0% NaCl.

In some embodiments, the co-solvent is a non-aqueous solvent. In some embodiments, the co-solvent is polyethylene glycol (PEG), ethanol, propylene glycol (PG), dimethylacetamide (DMA), dimethyl sulfoxide (DMSO), N-methyl-2-pyrrolidone (NMP), glycofurol, glycerol formal, SOLKETAL, acetone, tetrahydrofurfuryl alcohol (THFA), diglyme, dimethyl isosorbide, or ethyl lactate. In some embodiments, the co-solvent is polyethylene glycol, ethanol, propylene glycol, or dimethylacetamide. In some embodiments, the polyethylene glycol is PEG 200, PEG 300, PEG 400, PEG 500, PEG 600, PEG 700, PEG 800, PEG 900, or PEG 1000.

The term “active pharmaceutical ingredient” (API) as used herein refers to any component that is intended to furnish pharmaceutical activity or other direct effect in the diagnosis, cure, mitigation, treatment, or prevention of disease in humans or other animals. In some embodiments, the API is a small molecule. In some embodiments, the API is a macromolecule, e.g., a peptide or a protein, a polysaccharide, a lipid or a nucleic acid. Said active pharmaceutical ingredient is to be used in a pharmaceutical composition (pharmaceutical formulation) intended for parenteral administration to a human or an animal. In some embodiments, the formulation is an emulsion. In some embodiments, the formulation is a suspension, e.g., aqueous suspension.

In some embodiments, the API is poorly water soluble. The term “poorly water soluble” as used herein refers to compounds which have a solubility of, at room temperature and atmospheric pressure, less than about 1 mg/ml in water. In some embodiments, the solubility is less than or about 0.5 mg/mL. In some embodiments, the solubility is less than or about 0.1 mg/mL. In some embodiments, the solubility is less than or about 0.05 mg/mL. In some embodiments, the solubility is less than or about 0.01 mg/mL. In some embodiments, the solubility is less than or about 0.001 mg/mL. In some embodiments, solubility is determined at a pH of about 5.0, about 6.0, about 7.0, about 7.4, or about 8.0. The determination of water solubility is described by the United States Pharmacopeia. In some embodiments, the pharmaceutical formulation is an emulsion. In some embodiments, the pharmaceutical formulation is a suspension, e.g., an aqueous suspension.

In some embodiments, the pharmaceutical formulation comprises a viscous liquid. In some embodiments, the solvent is a viscous liquid. In some embodiments, the co-solvent is a viscous liquid. The term “viscosity” as used herein refers to the resistance of a fluid flow due to a shearing force. For example, a fluid with high viscosity will flow more slowly than a fluid with a low viscosity. Viscosity can be measured using a viscometer or a rheometer. In some embodiments, a viscous liquid has a viscosity of less than 1 mPa/s. In some embodiments, a viscous liquid has a viscosity of less than 0.5 mPa/s. In some embodiments, a viscous liquid has a viscosity of less than 0.1 mPa/s.

In some embodiments, methods of the present disclosure screen pharmaceutical formulations comprising a poorly water soluble API for tolerance using the assays described herein. In some embodiments, the method screens pharmaceutical formulations comprising a poorly water-soluble API for hemolysis ability.

In some embodiments, the human blood is whole human blood. Whole human blood refers to blood directly drawn from the body and from which none of the components such as plasma or platelets, have been removed. In some embodiments, the blood thinner is an anti-coagulant. In some embodiments, the blood thinner is heparin.

In some embodiments, erythrocytes are used as a surrogate marker for venous endothelial membranes. Thus, in some embodiments, hemolysis of erythrocytes indicates hemolysis at a venous endothelial membrane at the site of administration of a pharmaceutical formulation. In some embodiments, erythrocytes are separated from the second solution using centrifugation. Methods of separating erythrocytes can be selected by the skilled artisan. In some embodiments, the second solution is contained and subjected to centrifugation in a test tube.

In some embodiments, monitoring hemolysis comprises measuring the amount of hemoglobin. Hemoglobin may be measured, e.g., by adding Drabkin's solution to the sample and measuring the absorbance at 540 nm. Hemoglobin measurement is described in, e.g., Copeland et al., American Journal of Clinical pathology, 92 (5):619-624 (1989) and Mieczkowska et al., Anal Bioanal Chem 399:3293-3297 (2011).

In some embodiments, monitoring hemolysis comprises measuring hemolysis with different concentrations of the second solution. In some embodiments, a concentration of the second solution that exhibits lower than a pre-defined hemolysis threshold is used for administration to the patient. In some embodiments, the hemolysis threshold is less than 10% hemolysis. In some embodiments, the hemolysis threshold is less than 5% hemolysis. In some embodiments, the hemolysis is less than about 1% hemolysis.

In some embodiments, monitoring hemolysis comprises: diluting the second solution to a volume of about 200 μL to about 1000 μL, e.g., 400 μL into water or 0.9% NaCl, into containers; adding whole blood to container; homogenizing the components; incubating the containers for about 5 to about 30 minutes, e.g., about 10 minutes at about 25° C. to about 45° C., e.g., 37° C.; homogenizing the components again; centrifuging for about 5 minutes to about 30 minutes, e.g., about 10 minutes, at about 2500×g; removing about 10 μl to 100 μl, e.g., about 30 μl of the solution from each container into the wells of a plate; adding about 100 μl to about 500 μl, e.g., about 170 μl of Drabkin's solution to the wells containing sample; and reading the absorbance of each well at 540 nm.

In some embodiments, the container is a glass test tube or a vial. In some embodiments, the container comprises a material that is resistant to degradation, e.g., dissolution, by the solvent and/or the co-solvent. In some embodiments, the container comprises a material that does not absorb the solvent and/or the co-solvent. In some embodiments, the container does not comprise a plastic. Non-limiting examples of plastics include polycarbonate, polyethylene, (e.g., high density polyethylene, low-density polyethylene, polyethylene terephthalate), polypropylene, polystyrene, (e.g., high impact polystyrene), polyurethane, polyvinyl chloride, acrylonitrile butadiene styrene, and the like. In some embodiments, the solvent and/or co-solvent is capable of degrading plastic, e.g., dissolving plastic. In some embodiments, the solvent and/or co-solvent is capable of being absorbed by plastic. In some embodiments, degradation or absorption of the solvent and/or co-solvent by the plastic reduces the ability of the container to maintain its shape. In some embodiments, degradation or absorption of the solvent and/or co-solvent by the plastic reduces the ability of the container to hold the contents therein.

In some embodiments, the container has the capacity of greater than or about 200 μL, greater than or about 300 μL, greater than or about 400 μL, greater than or about 500 μL, greater than or about 600 μL, greater than or about 700 μL, greater than or about 800 μL, greater than or about 900 μL, or greater than or about 1 mL. In some embodiments, the container has a capacity of about 1 mL, and the second solution is diluted to a volume of about 400 μL in the container. The containers described herein advantageously allow for mixing of the components, e.g., the second solution and whole blood, without degrading or being absorbed by the container. The high capacity of the containers described herein also allow for mixing of high viscosity liquids, which cannot be achieved in containers having a small volume capacity e.g., wells of a multi-well plate.

In some embodiments, one or more controls are used for screening hemolysis ability of the second solution. In some embodiments, a control that exhibits 100% hemolysis and a control that exhibits 0% hemolysis are also measured in the same plate as the dilutions of the second solution. In some embodiments, a control with 100% hemolysis, 0% hemolysis, less than or about 5% hemolysis, greater than or about 10% hemolysis, and greater than or about 50% hemolysis are also measured in the same plate as the dilutions of the second solution.

The percent hemolysis can be measured and plotted on a curve (e.g. by using commonly known software). The maximum acceptable percent hemolysis can be used to determine the appropriate concentration of test solution (e.g. by using commonly known software). In such embodiments, erythrocytes are used as a surrogate marker of venous endothelial membranes.

In some embodiments, the in vitro method for testing tolerance of substance or combination of substances to be used in a liquid pharmaceutical composition intended for parenteral administration to an animal is a miscibility test. “Miscibility” refers to the ability of two substances to mix in all proportions and form a homogeneous solution. For example, two substances are “miscible” if they form one phase upon mixing, and no distinct layers are visible. Water and ethanol are examples of miscible liquids. “Immiscible” substances remain in two different phases when mixed, e.g., oil and water. Contacting two immiscible substances may result in precipitation of one or both of the substances, or precipitation of compounds soluble in one or both of the substances.

In some embodiments, the pharmaceutical formulation comprises a small molecule compound, e.g., a small molecule API. In some embodiments, contacting the pharmaceutical formulation comprising a poorly water-soluble API with an immiscible substance causes precipitation, e.g., of the poorly water-soluble API. In some embodiments, increasing miscibility of a pharmaceutical formulation with plasma provides local tolerance.

In some embodiments, the present disclosure provides an in vitro method of screening a pharmaceutical formulation comprising an API, a solvent and co-solvent for miscibility with plasma, comprising: (a) contacting the formulation with a solution comprising plasma to produce a second solution; and (c) monitoring precipitation in the second solution.

In some embodiments, the solvent is water. In some embodiments, the solvent comprises a salt. In some embodiments, the solvent comprises NaCl. In some embodiments, the solvent comprises between about 0.1% to about 20% NaCl. In some embodiments, the solvent comprises between about 0.5% to about 2% NaCl. In some embodiments, the solvent comprises about 0.1%, about 0.2%, about 0.3%, about 0.4%, about 0.5%, about 0.6%, about 0.7%, about 0.8%, about 0.9%, or about 1.0% NaCl.

In some embodiments, the co-solvent is a non-aqueous solvent. In some embodiments, the co-solvent is polyethylene glycol (PEG), ethanol, propylene glycol (PG), dimethylacetamide (DMA), dimethyl sulfoxide (DMSO), N-methyl-2-pyrrolidone (NMP), glycofurol, glycerol formal, SOLKETAL, acetone, tetrahydrofurfuryl alcohol (THFA), diglyme, dimethyl isosorbide, or ethyl lactate. In some embodiments, the co-solvent is polyethylene glycol, ethanol, propylene glycol, or dimethylacetamide. In some embodiments, the polyethylene glycol is PEG 200, PEG 300, PEG 400, PEG 500, PEG 600, PEG 700, PEG 800, PEG 900, or PEG 1000.

In some embodiments, methods of the present disclosure screen pharmaceutical formulations comprising poorly water-soluble API for tolerance using the assays described herein. In some embodiments, the method screens pharmaceutical formulations comprising a poorly water-soluble API for hemolysis. In some embodiments, the method screens pharmaceutical formulations comprising a poorly water-soluble API for miscibility with plasma.

In some embodiments, the plasma is obtained from whole blood. In some embodiments, the plasma is obtained from heparinized human blood. In some embodiments, the plasma is obtained by centrifugation of heparinized human blood.

In some embodiments, precipitation is monitored by using light scattering. In some embodiments, precipitation is monitored using a turbidity sensor or turbidimeter or nephelometer. In some embodiments, precipitation is monitored using a spectrophotometer. In some embodiments, precipitation occurs when the formulation is immiscible with plasma; immiscibility with plasma increases the size of the injection bolus which is to be avoided.

All references herein, including patents, patent applications, papers, text books, and the like, and the references cited therein, to the extent that they are not already, are hereby incorporated herein by reference in their entirety.

### EXAMPLES

### Example 1. Hemolysis Test

A test was developed to determine the hemolytic activity of a pharmaceutical formulation. The test utilized fresh anticoagulated human blood. In general, the test was performed by diluting the pharmaceutical product with fresh saline, then mixing with an aliquot of fresh human whole blood. After incubating at 37° C., the test solutions were centrifuged, and the concentration of hemoglobin was measured in the supernatant using spectroscopic techniques.

List of Equipment, Materials, and Reagents

The equipment used in this example included: biosafety cabinet (e.g., BERNER CLAIRE PRO C-3-190); centrifuge (e.g., EPPENDORF 5920R); incubator (e.g., MEMMERT 130); plate reader (e.g., MOLECULAR DEVICES SPECTRAMAXID3).

The materials used in this Example included: test tubes (e.g., FALCON Cat No. 352057; LONZA Cat No. N207); flat-bottom transparent multi-well plates (e.g., CORNING Cat No. 9017); heparin tubes (e.g., Lithium Heparin tubes, BD VACUTAINER Cat No. 367880).

The reagents used in this Example included: 0.9% NaCl (e.g., NaCl 0.9%) sterile, pyrogen-free (B.BRAUN Cat No. 190/12606051/0812); distilled water (e.g., LAL reagent water, LONZA Cat No. W50-100; water for injection, B.BRAUN Cat No. 530101); ethanol (e.g., SIGMA Cat. No. 51976-500ML-F); Drabkin's Reagent (e.g., SIGMA Cat. No. D5941); BRIJ L23 (e.g., SIGMA Cat. No. B4184); human blood (e.g., heparinized whole blood samples from humans).

The procedure for the preparation of Drabkin's solution was performed: one vial of Drabkin's reagent was reconstituted with 1000 ml water. Then, 0.5 ml of 30% BRIJ L23 Solution was added to the Drabkin's solution. The mixture was mixed well and filtered if insoluble particles remained. The solution was stored protected from light at room temperature for up to 6 months.

To determine the 100% hemolysis value, equal volumes of saline and blood were mixed, centrifuged, and mixed again. 30 μl was transferred to a plate, and 170 μl of Drabkin's reagent was added. The 100% hemolysis value was indicated as Value A.

To determine the 0% hemolysis value, equal volumes of saline and blood were mixed. The 0% hemolysis value is indicated as Value B.

Test controls were treated similarly as the test samples:

Control 1: 20% of ethanol in saline, expected to have <5% of hemolysis;

Control 2: 30% of ethanol in saline, expected to have >10% of hemolysis;

Control 3: distilled water or water for injection; expected to have >50% of hemolysis.

The following dilutions of test substance in saline was chosen: 1:1 (as is), 1:2, 1:4, 1:8, and 1:16. The series may be continued if strong hemolysis is expected. These values are indicated as Values C.

Standard Procedure

In a standard procedure, NaCl 0.9% sterile (saline) was used as a diluent. The dilutions were preformed directly in a 96-well plate. Preferably, round bottom microplates were used. Each well was filled with 200 μl of test sample. The sample was diluted by transferring 100 μl from well 1 to well 2, mixing using a pipette, and continuing the dilution to well 3, etc. 100 μl of the last well was discarded. Each dilution was performed in triplicates.

100 μl of heparinized blood was added to all dilutions and test controls including the 0% determination of hemolysis. The plate was shaken carefully on a plate shaker and incubated for 10 minutes at 37° C. The plate was shaken again, then centrifuged at approximately 2500×g for 10 minutes. The wells of Value A were mixed for 100% hemolysis. 30 μl of each well was transferred to a new 96-well plate (flat bottom), and 170 μl of Drabkin's solution was added to each well containing test solution. The plate was read in a plate reader at wavelength 540 nm.

Study Procedure

In the study procedure, for example, for viscous liquids, saline was used as described for the standard procedure to dilute the product (e.g., the solvent) to the following concentrations: 0%, 30%, 40%, 50%, 55%, 60%, 70%, 85%, and 100%.

The dilutions were performed in glass or plastic tubes with a diameter of approximately 12 mm and a length of approximately 100 mm. The controls were prepared in a similar manner as above for the standard procedure (100% hemolysis and 0% hemolysis).

An exemplary dilution table is shown below:

| | Conc. | | | | | | | | |
|---|---|---|---|---|---|---|---|---|---|
| | 0% | 30% | 40% | 50% | 55% | 60% | 70% | 85% | 100% |
| Solvent | 0 | 120 | 160 | 200 | 220 | 240 | 280 | 340 | 400 |
| μl | | | | | | | | | |
| Diluent | 400 | 280 | 240 | 200 | 180 | 160 | 120 | 60 | 0 |
| μl | | | | | | | | | |

The diluent and concentrations may be modified.

400 μl of whole blood was added to each tube. The components were carefully homogenized. The tubes were incubated for 10 minutes at 37° C., homogenized again and then centrifuged for 10 minutes at 2500×g. The contents of the tube were mixed for Value A (100% hemolysis) and then transferred 3 times 30 μl of each tube of the supernatant to a 96-well plate (flat bottom) and 170 μl of Drabkin's solution was added to each well containing solution. The plate was read in a plate reader at wavelength 540 nm.

The test may be adjusted to fit the following parameters:

The mean of the 100% hemolytic value (Value A) is about 200 times higher than the mean of the 0% hemolytic value (Value B);

Control 1 has less than 5% hemolytic activity:

Control 2 has more than 10% hemolytic activity:

Control 3 has more than 50% hemolytic activity;
A test formulation is not hemolytic if the hemolytic activity of the undiluted test solution is below 5%.

For evaluation of the test results from the plate reader reading at wavelength 540 nm, the mean values of the triplicates were calculated. The percent hemolytic activity was determined according to the following formula:

$$\% HA = \frac{C - B}{A - B} \times 100$$

wherein: % HA=percent hemolytic activity; A=Value for 100% HA; B=Value for 0% HA; C=Value for tested solution.

### Example 2. Hemolysis Test with Various Co-Solvents

The tested co-solvents in this Example were:
Polyethylene glycol 300 (PEG 300) (SIGMA Cat. No. 90878-250ML-F, Lot BCBT7971);
Polyethylene glycol 400 (PEG 400) (SIGMA Cat No. 91893-250ML-F, Lot BCBV5160);
Ethanol (SIGMA Cat No. 51976-500ML-F, Lot BCBW5361);
Propylene glycol (PG) (SIGMA Cat No. 892280-250ML-F, Lot BCBV5491);
Dimethylacetamide (DMA) (SIGMA Cat. No. 270555-1L-F, Lot SHBJ3324).

Reagents: the water used in the experiments described in this example was from LONZA, sterile, pyrogen-free (Lot 18183408); the saline used in the experiments described in this Example was from B.BRAUN, sterile, pyrogen-free (Lot 17487406).

The hemolysis test was performed according to Example 1. Dilutions of the co-solvents were prepared in tubes. An equal volume of human heparinized whole blood was added and homogenized. The test co-solvents were diluted in sterile, pyrogen-free water and sterile, pyrogen-free saline.

The tested concentrations were 10%, 20%, 30%, 40%, 50%, 55%, 60%, 70%, 85%, and 100% v/v.

The water-only control showed hemolysis values of between 84.3% and 92.8% hemolysis, indicating the tests were valid. The co-solvents ethanol, dimethylacetamide, and propylene glycol diluted in water generated hemolysis values close to 100% in the lowest tested concentration of 10%.

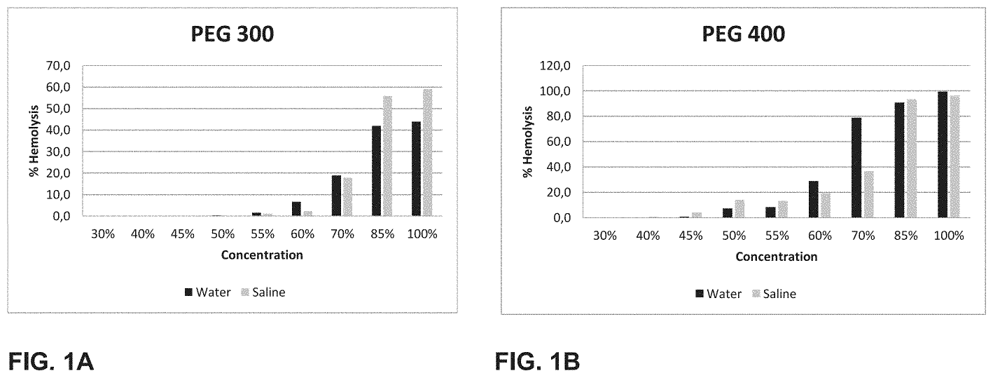

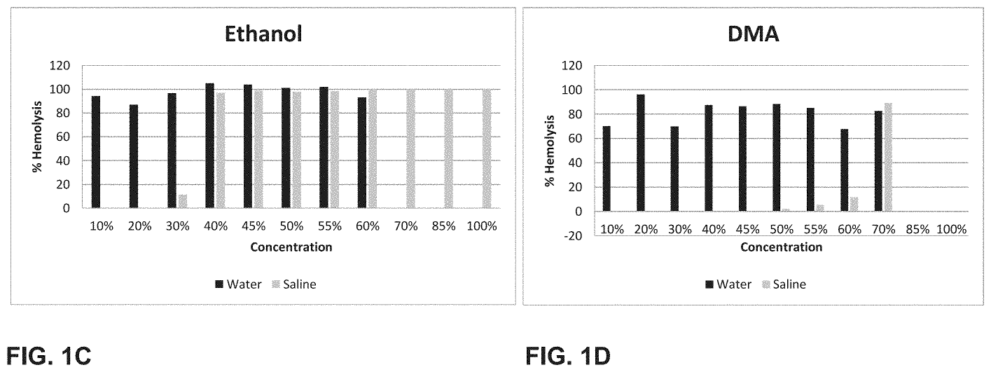

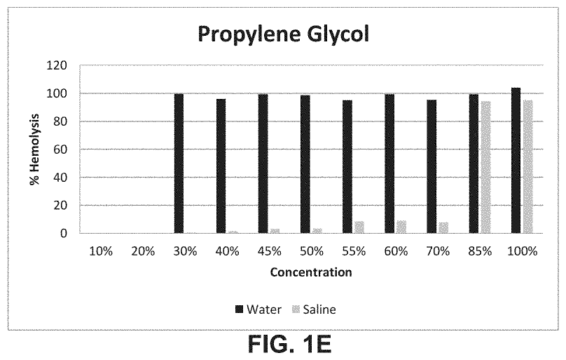

Results shown in Table 1 and FIGS. 1A-1 E:

PEG 300 induced a significant (>10% hemolysis) at co-solvent concentrations between 60% and 70%. For PEG 400, the >10% hemolysis occurred at between 50% and 60% co-solvent. For both PEG 300 and PEG 400, the switch from water for injection to saline as the diluent did not drastically modify the hemolytic behavior of the co-solvent. All four curves followed a sigmoidal, S-shaped curve.

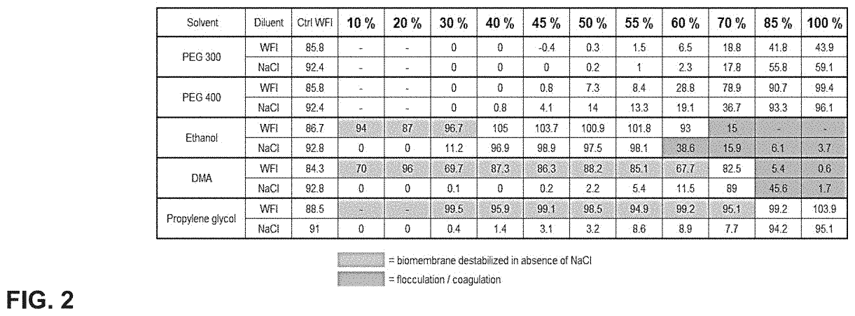

For ethanol, DMA, and PG, high hemolysis was observed at the lowest tested co-solvent concentration (10%). This was unexpected because the solutions with 10% co-solvent are close to isotonicity (PEG 400), isotonic (PEG 300), or clearly hypertonic (DMA, PG, and ethanol). See Table 2 and FIG. 2.

Sodium chloride appeared to have a stabilizing effect on the biomembrane, which was surprising because sodium chloride may destabilize colloidal solutions or suspensions by minimizing the zeta potential or reducing viscosity of protein solutions by reduction of electrostatic interactions. Sodium chloride was described to have a stabilizing effect on erythrocyte membrane. Lenos et al., Cell Biochem Biophys 61 (3):531-537 (2001).

Without binding to a particular theory, one explanation for the stabilizing effect of sodium chloride may relate to the effect of sodium chloride on lipid bilayers. Böckmann et al., Biophys J 85:1647-1655 (2003). The study based on fluorescence correlation spectroscopy experiments and molecular dynamics simulations has pointed out the role of monovalent ions in organizing the membrane.

When taking into consideration the surface charge of a lipid bilayer, a role emerges for the counter ion of the phosphate groups (e.g., Na⁺). While neutral phospholipids such as sphingomyelin and zwitterionic phosphatidylcholine are located primarily in the outer leaflet of the plasma membrane, most anionic phospholipids, such as phosphatidic acid (PA), phosphatidylserine (PS), phosphatidylethanolamine (PE) and phosphatidylinositol (PI) species, such as phosphatidylinositol 4,5-bisphosphate (PIP2) and phosphatidylinositol (3,4,5)-trisphosphate are mostly located at the inner leaflet. Ma et al., Front Immunol 8:1513 (2017). The low acid dissociation constant (pKa) values of the phosphate groups of the lipid head group are responsible for the negative charge of these lipids at physiological pH. In the presence of plasma, the negatively charged phosphate groups at the external surface of the biomembrane are essentially neutralized by a counter-ion (e.g., Na⁺). Therefore, any washing-out effect of the counter-ion would locally modify the electronic density. The consecutive repulsive electrostatic forces generate a slight destabilization of the biomembrane, which would be sufficient to favor hemolysis in a static in vitro test, where the test solution is mixed with blood and kept at 37° C. for 10 minutes. In a dynamic situation, e.g., during a clinical administration, a co-solvent solution would be rapidly diluted by circulating blood. Therefore, to avoid test artifacts, the in vitro hemolysis test can be performed with co-solvent formulations diluted with an aqueous NaCl solution (e.g., NaCl 0.9%) and not water for injection.

Another observation from the test results was significant flocculation with ethanol and DMA solutions at higher co-solvent concentrations, as shown in Table 1. Since the flocculation occurred with both water and saline dilutions, a different, non-binding explanation is provided.

Albumin has an isoelectric point (pI) of 4.9. If proteins with similar pI protrude from the biomembrane of the erythrocytes, the proteins would be negatively charged at physiological pH. It has been demonstrated of the surface charge of the red blood cells inhibits their aggregation. Jan et al., Gen Physiol 61 (5):638-654 (1973). The isoelectric point of a protein is the result of all pKa's of the individual residues of the protein. Since the pKa generally increases with increasing concentration of the co-solvent, the pI would shift to higher values. Once the pI is sufficiently close to physiological pH, the zeta potential will be small enough to enable flocculation. For a hemolysis test in which the hemolysis threshold is low, e.g., 5%, flocculation occurring only at high co-solvent concentrations would not impact the test.

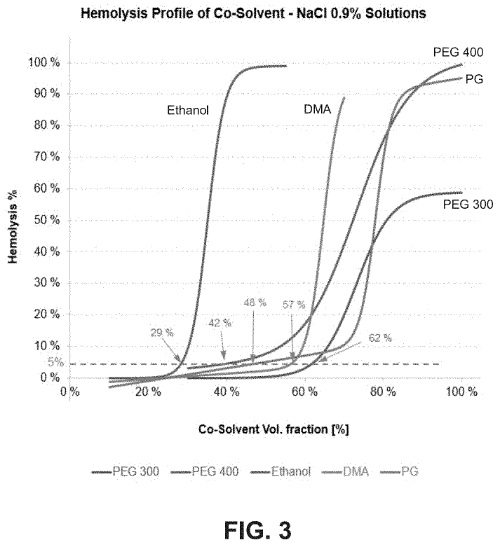

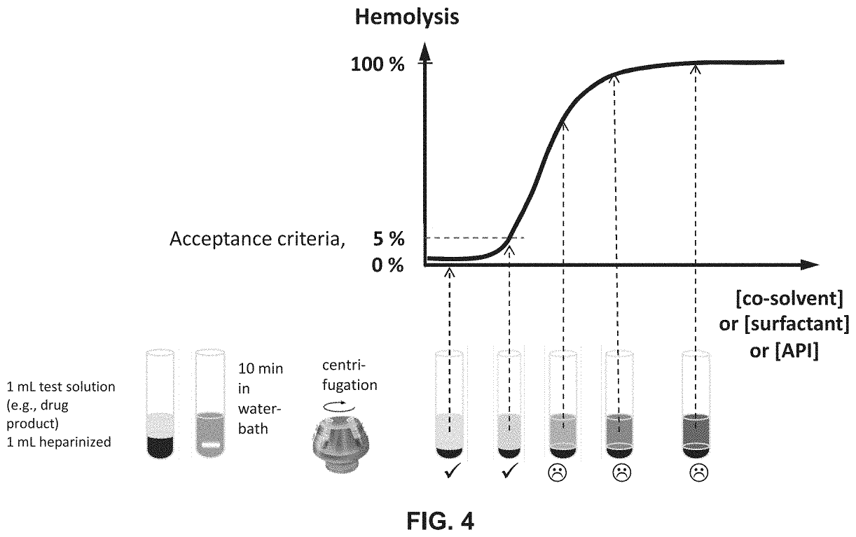

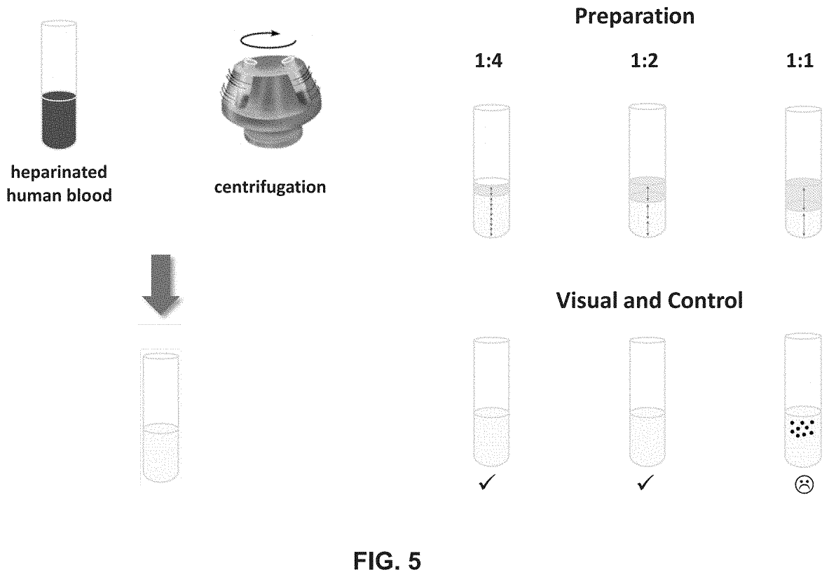

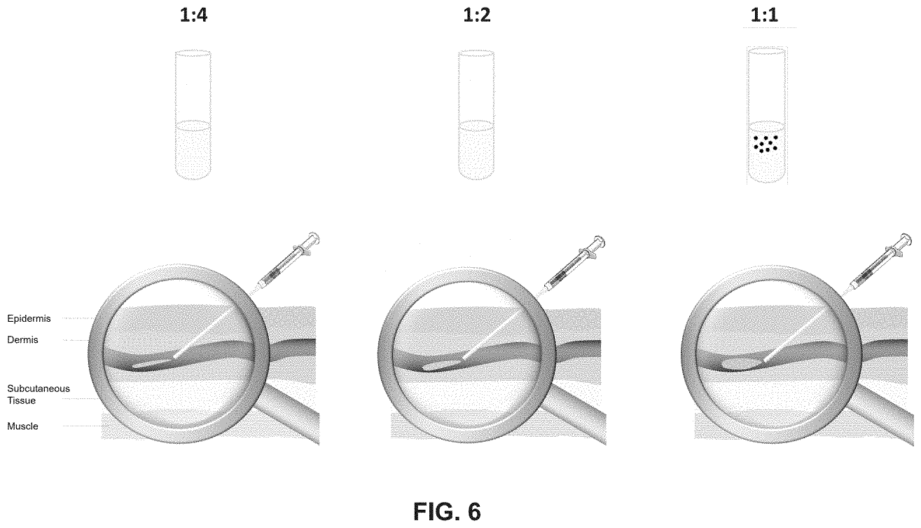

Based on the obtained hemolysis test results with solvent dilution in NaCl 0.9%, the highest possible concentration for each co-solvent was determined. See FIG. 3. The following equation was used to obtain a curve fitting of the hemolysis data:

$$y = \frac{B}{1 + {e}^{( - k \times ( x - A ) )}} + C x + D$$

Values for the various parameters of the equation above are indicated in Table 3.

TABLE 3

| | PEG 300 | PEG 400 | Ethanol | DMA | PG |
|---|---|---|---|---|---|
| k = | 0.2200 | 0.1500 | 0.4500 | 0.4820 | 0.5000 |
| A = | 73.0000 | 73.0000 | 35.0000 | 64.6628 | 78.0000 |
| B = | 59.0000 | 91.0000 | 99.0000 | 91.4094 | 80.0000 |
| C = | — | 0.1000 | — | 0.0871 | 0.2000 |
| D = | — | 0.0000 | — | −2.1583 | −5.0000 |
| max. well tolerated co-solvent concent | 62% | 42% | 29% | 57% | 48% |

The maximal well tolerated co-solvent concentration was obtained by calculating the intersection of the sigmoidal curves with the horizontal line at hemolysis, i.e., 5%.

1. A method for in vitro determination of blood tolerance for a substance or combination of substances to be used in a liquid pharmaceutical composition intended for parenteral administration to an animal as a surrogate test for animal and/or human testing, comprising conducting a miscibility test with blood plasma obtained from whole blood or from heparinized blood.

2. The method according to claim 1, wherein the substance to be tested is selected from active pharmaceutical ingredients, organic solvents, co-solvents, surfactants, and combinations of two or more thereof, wherein the active pharmaceutical ingredient is poorly water soluble.

3. The method according to claim 1, wherein the animal to which the pharmaceutical composition is to be administered parenterally is a mammal, selected from humans, domestic animals and productive livestock.

4. The method according to claim 1, wherein a plurality of concentrations of the substance is tested for precipitation or coagulation in blood.

5. The method according to claim 1, wherein the lowest concentration when precipitation or coagulation first occurs is determined.

6. The method according to claim 4, wherein the plurality of concentrations is obtained by varying the mixing ratio of (a) blood plasma and (b) the substance to be tested if the substance is liquid at room temperature or a solution of the substance to be tested if the substance is a solid at room temperature.

7. The method according to claim 4, wherein precipitation or coagulation is detected by light scattering, a turbidity sensor, a turbidimeter, a nephelometer or a spectrophotometer.

8. The method according to claim 5, wherein precipitation or coagulation is detected by light scattering, a turbidity sensor, a turbidimeter, a nephelometer or a spectrophotometer.
